(19) United States
(12) Patent Application Publication
(10) Pub. No.: US 2019/0216603 A1
(43) Pub. Date: Jul. 18, 2019

(54) APPARATUS AND METHOD FOR ACCESSING AORTA

(71) Applicant: TEL HASHOMER MEDICAL RESEARCH, INFRASTRUCTURE AND SERVICES LTD., Ramat Gan (IL)

(72) Inventors: Boris ORLOV, Rishon LeZion (IL); Ehud RAANANI, Hod Hasharon (IL)

(21) Appl. No.: 16/313,166

(22) PCT Filed: Jun. 29, 2017
(86) PCT No.: PCT/IL2017/050726

§ 371 Date: Dec. 26, 2018

Related U.S. Application Data
(60) Provisional application No. 62/356,545, filed on Jun. 30, 2016.

(51) Int. Cl. A61F 2/24; A61B 17/34; A61B 17/12

(52) U.S. Cl. CPC A61F 2/2466; A61B 17/3423; A61B 17/12109; A61B 2017/00243; A61F 2/2433; A61B 17/12136; A61B 17/12036

(57) ABSTRACT

A method of accessing the ascending aorta via the superior vena cava (SVC) comprising puncturing the adjacent wall regions of the SVC and AA to provide passage from the SVC into the AA.

### RELATED APPLICATIONS

This application claims benefit under 35 U.S.C. 119(e) of U.S. Provisional Applications 62/356,545, filed Jun. 30, 2016, the disclosure of which is incorporated herein by reference.

### TECHNICAL FIELD

Embodiments of the disclosure relate to cardiac surgery and cardiac valve prosthesis.

### BACKGROUND

The human heart comprises two blood pumps that operate in synchrony to oxygenate and deliver oxygenated blood to the body. A first pump receives low oxygenated venous blood from the various parts the body, and pumps the blood through the lungs to be oxygenated. The second pump receives the oxygenated blood from the lungs and pumps it to flow through the systemic arteries of the circulatory system to deliver oxygen and nutrients to the body parts. The two pumps are located adjacent each other in the heart and each pump comprises two chambers, an atrium that receives blood and a ventricle that pumps blood.

The first pump is located on the right side of the heart and comprises the right atrium and right ventricle. The second pump is located on the left side of the heart and comprises the left atrium and left ventricle of the heart. Cardiac valves referred to as the tricuspid and pulmonary valves control direction of blood flow in the right side of the heart. The tricuspid valve is located between the right atrium and right ventricle. The pulmonary valve is located between the right ventricle and the pulmonary artery. Low oxygenated venous blood enters the right atrium from the superior vena cava (“SVC”) and the inferior vena cava (IVC), and during a part of the heart cycle referred to as diastole the right ventricle relaxes, the tricuspid valve opens, and the blood flows through tricuspid valve from the right atrium into the right ventricle. During a subsequent part of the heart cycle referred to as systole the tricuspid valve closes, the pulmonary valve opens and the right ventricle contracts to pump the low oxygenated venous blood that it received from the right atrium out of the ventricle and into the pulmonary artery via the pulmonary valve for oxygenation in the lungs. Cardiac valves referred to as the mitral and aortic valves operate to control direction of blood flow in the left side of the heart. Oxygenated blood from the lungs enters the left atrium via the pulmonary veins. During diastole the oxygenated blood flows via the mitral valve from the left atrium into the left ventricle. The left ventricle contracts during systole to pump the oxygenated blood that it received from the left atrium out of the heart through the aortic valve and into the ascending aorta (“AA”), via the aortic valve for delivery to the body.

Each cardiac valve comprises a set of matching “flaps”, also referred to as “leaflets” or “cusps”, which are mounted to and extend from a supporting ring structure of fibrous tissue, referred to as the annulus of the valve. The leaflets are configured to align and overlap each other, or coapt, along free edges of the leaflets to close the valve. The valve opens when the leaflets are pushed away from each other by positive blood pressure in the desired flow direction and their free edges part.

Efficient cardiac function can be complex and cardiac valve and/or muscle may become compromised by disease or injury to an extent that warrants surgical intervention to effect repair or replacement to provide a person suffering from cardiac malfunction with an acceptable state of health and quality of life. For example, a patient may require surgical replacement of a native heart valve with an artificial heart valve to restore proper blood flow in the heart.

### SUMMARY

An aspect of an embodiment of the disclosure relates to providing a method of deploying an aortic valve prosthesis from a peripheral vein.

The aortic valve prosthesis deployment method in accordance with an embodiment of the disclosure, which may be referred to herein as a Transcaval Transaortic Transcatheter Aortic Valve Implantation (“3T-AVI”) method, comprises: guiding an inflatable balloon connected to a first tube and a first guidewire to a portion of the superior vena cava (SVC) that is adjacent to the ascending aorta (AA); positioning and inflating the balloon at the SVC portion so that a needle port comprised on an outer surface of the balloon is securely positioned on an interior side of a wall of the SVC portion; extending a needle through the needle port to outside the needle port so that the needle creates a puncture traversing the SVC portion wall and an adjacent region of the wall of the AA; inserting a second guidewire from the SVC through the balloon and the puncture into the AA, and through the native aortic valve into the left ventricle; deflating the balloon; and withdrawing the first tube, balloon and first guidewire from the SVC while maintaining the second guidewire in the puncture and traversing the SVC, AA and aorta.

In an embodiment, the 3T-AVI method further comprises: guiding via the second guidewire a distal end of a second tube from the SVC through the puncture into the AA, the second tube having an approximation device (AD) loaded in a collapsed state near the distal end; ejecting a first portion of the AD from the distal end in the AA so that the first portion expands from the collapsed state to form an aortic anchor, which, when expanded operates to prevent the AD from being pulled out of the aorta; ejecting a second portion of the AD from the distal end within the puncture so that the second portion expands from the collapsed state to form a tubular junction optionally referred to as a bridge connected to the aortic anchor; ejecting a third portion of the AD from the distal end in the SVC so that the third portion expands from the collapsed state to form a venous anchor which, when expanded operates to maintain walls of the vein and AA in close spatial approximation and prevent the AD from being pulled out of the vein and into the aorta; and withdrawing the second tube from the SVC; wherein the tubular bridge in the expanded state deployed in the puncture is shaped and dimensioned to be narrow relative to the aortic anchor in the expanded state and the venous anchor in the expanded state, and forms a through hole connecting a first opening situated in the SVC and a second opening situated in the AA.

It is noted that the bridge is configured to have a length that facilitates operation of the venous and AA clamps in maintaining the walls of the vein and AA in close spatial approximation. It is additionally noted that the bridge is advantageously configured to enable passage through the bridge hole of a device for which introduction into the AA may be desired. For example, the bridge may be configured having a hole sufficiently large or elastically expandable to enable passage of the device.

In an embodiment of the disclosure, the 3T-AVI method further comprises: guiding via the second guidewire a distal end of a third tube from the SVC through the puncture into the AA and through the native aortic valve into the left ventricle, the third tube having a prosthetic aortic valve (PAV) loaded in a collapsed state near the distal end; and ejecting the PAV from the distal end at the native aortic valve, the PAV expanding into an expanded state from the collapsed state following ejection; and withdrawing the third tube from the left ventricle, the aortic valve, the AA and the puncture

In an embodiment of the disclosure, the 3T-AVI method further comprises closing the through hole comprised in the tubular bridge. Optionally, closing the through hole comprises inserting a plug into the through hole. Optionally, the plug is loaded into the second tube and inserted into the through hole via the distal end of the second tube.

In an embodiment of the disclosure, the balloon is shaped and dimensioned so that, in the inflated state, the balloon: is shaped to form a seal in the interior of the SVC portion sufficient to block blood flow between the exterior of the balloon and the interior wall of the SVC; and comprises a “passageway”, a “through hole” connecting a first opening on a downstream end of the inflated balloon and a second opening on an upstream end of the inflated balloon, the through hole being shaped and dimensioned to allow blood flow through the through hole.

In an embodiment of the disclosure, the aortic anchor, the venous anchor and/or the bridge comprises a wire mesh. Optionally, the wire mesh comprises nitinol.

An aspect of an embodiment of the disclosure relates to providing an approximation device (AD) comprising: a collapsible and expandable aortic anchor; a collapsible and expandable venous anchor; and a collapsible and expandable tubular bridge connected to the aortic anchor and the venous anchor, wherein the tubular bridge in the expanded state is: shaped and dimensioned to be narrow relative to the aortic anchor in the expanded state and the venous anchor in the expanded state; and comprises a channel connecting a first opening situated the aortic anchor and a second opening situated in the vein.

An aspect of an embodiment of the disclosure relates to providing an inflatable balloon for inserting into a blood vessel, the balloon comprising: a tubular wall comprising an outer surface and an inner surface, the inner surface surrounding a first through hole having a first opening at a first end of the balloon and a second opening at a second end of the balloon; and a second through hole traversing a portion of the tubular wall, the second through hole comprising first opening at the first end and a puncture port at an outer surface of the balloon wall.

In an embodiment of the disclosure, the inflatable balloon further comprising a third through hole traversing a portion of the tubular wall, the third through hole comprising a first opening at the first end and a second opening at the second end.

In the discussion, unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the description and claims is considered to be the inclusive “or” rather than the exclusive or, and indicates at least one of, or any combination of items it conjoins.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF FIGURES

Non-limiting examples of embodiments of the invention are described below with reference to figures attached hereto that are listed following this paragraph. Identical features that appear in more than one figure are generally labeled with a same label in all the figures in which they appear. A label labeling an icon representing a given feature of an embodiment of the invention in a figure may be used to reference the given feature. Dimensions of features shown in the figures are chosen for convenience and clarity of presentation and are not necessarily shown to scale.

FIGS. 1A-1K schematically show a 3T-AVI method for deployment of a PAV in the aortic valve of a subject, in accordance with an embodiment of the invention; and

FIGS. 2A-2B schematically show an approximation device in accordance with an embodiment of the invention.

### DETAILED DESCRIPTION

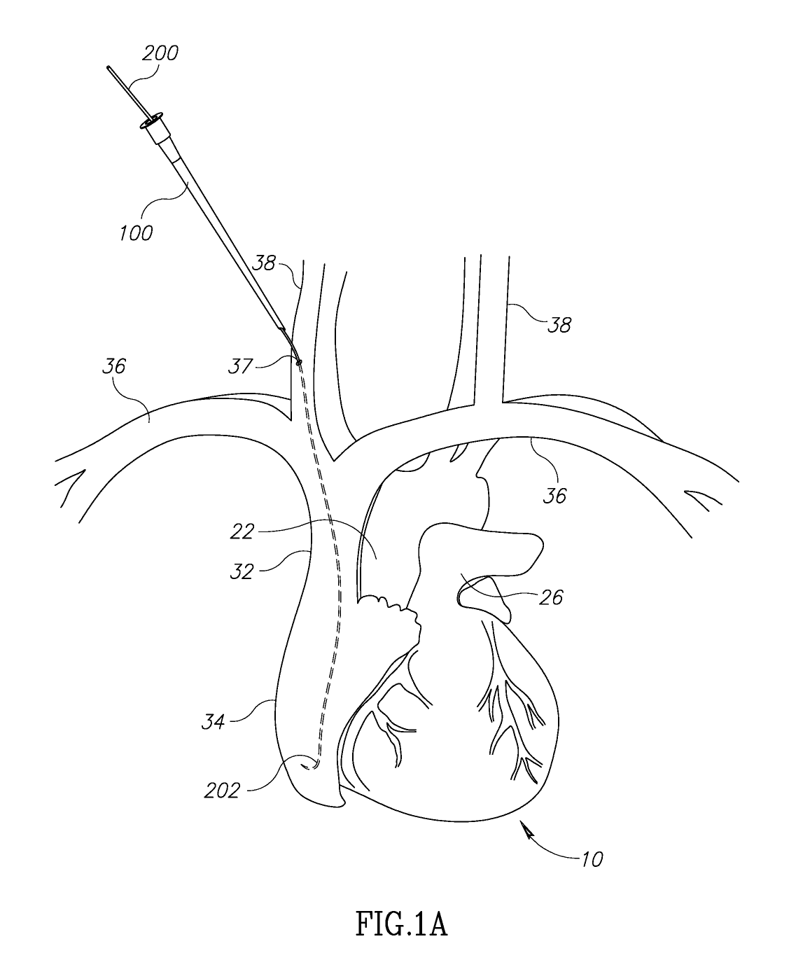

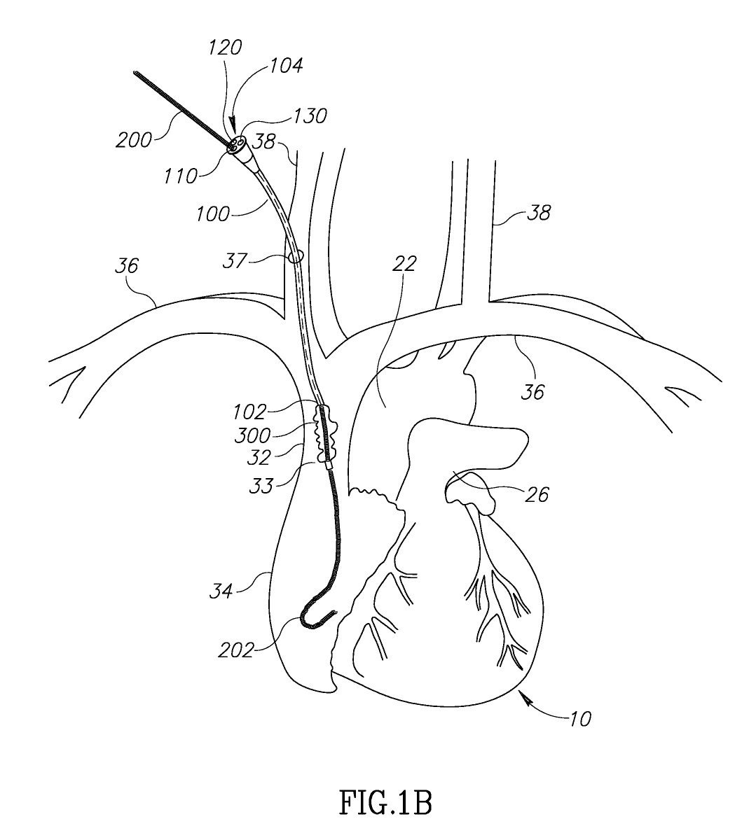

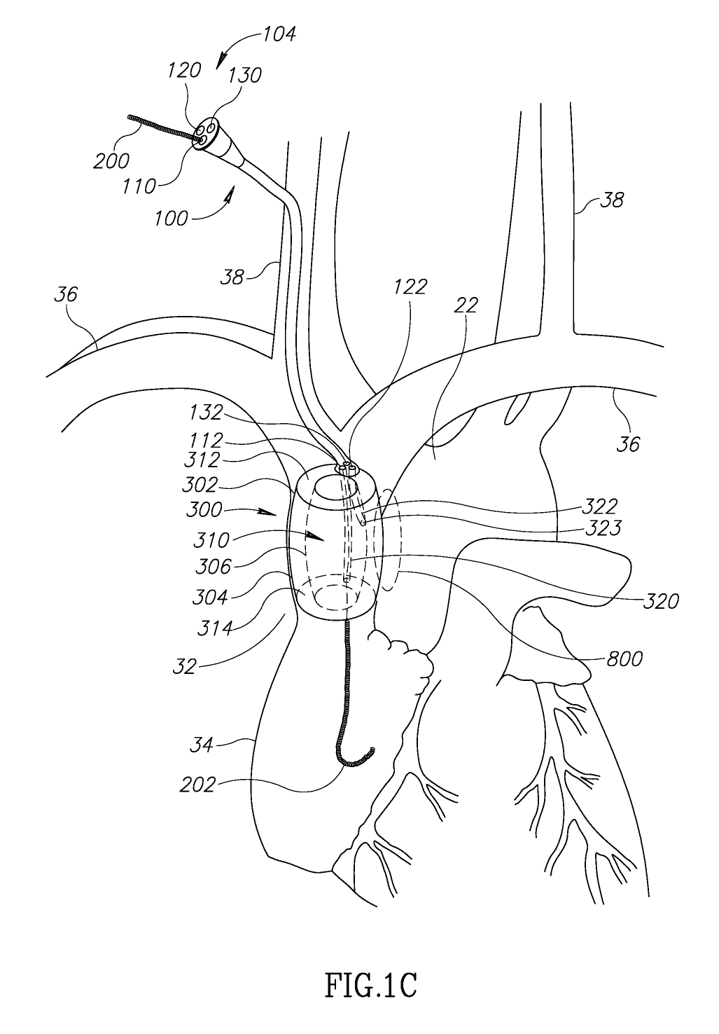

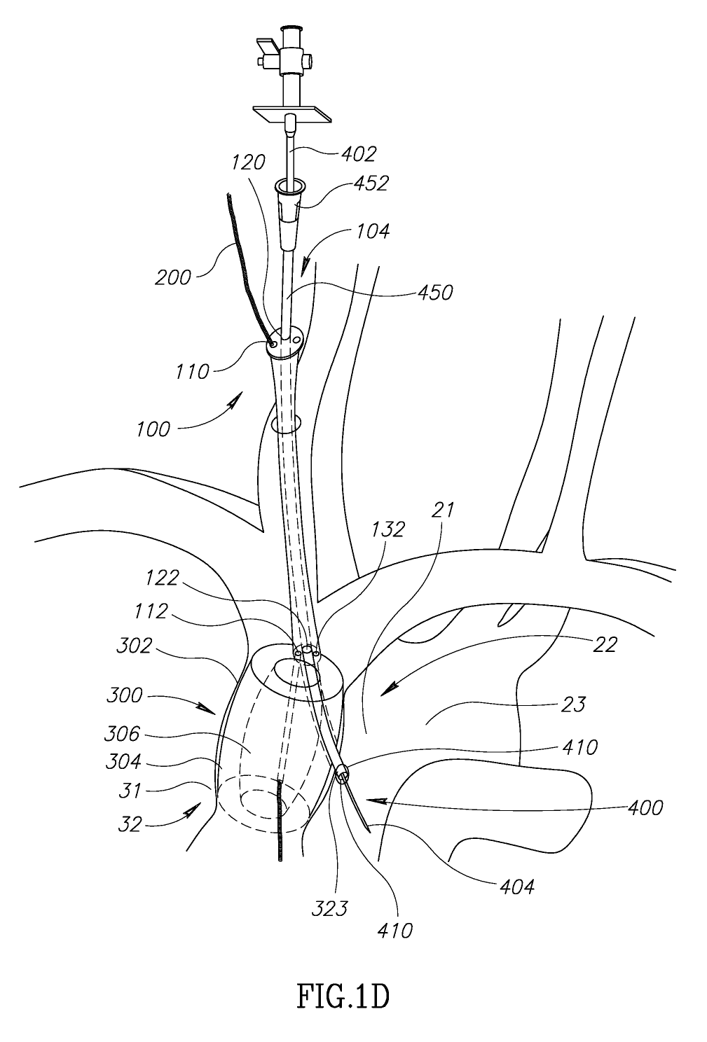

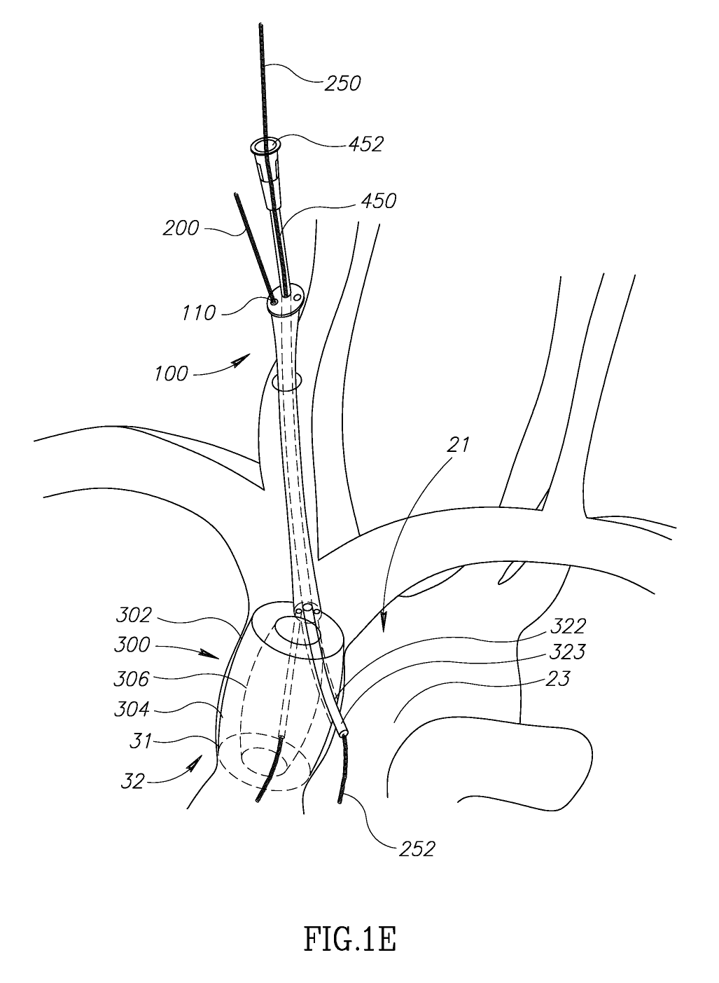

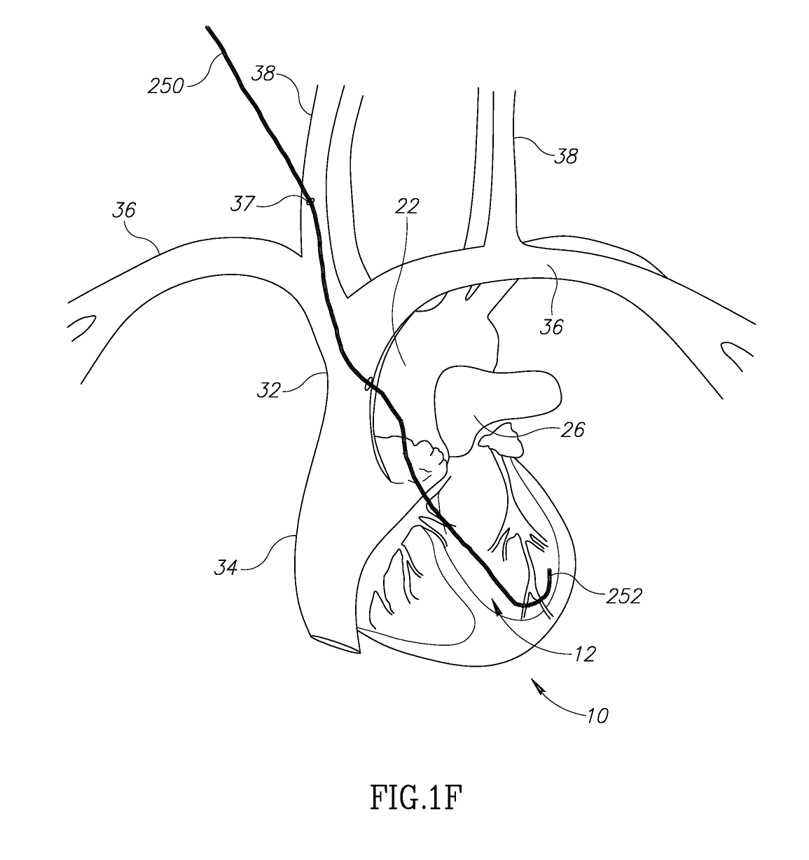

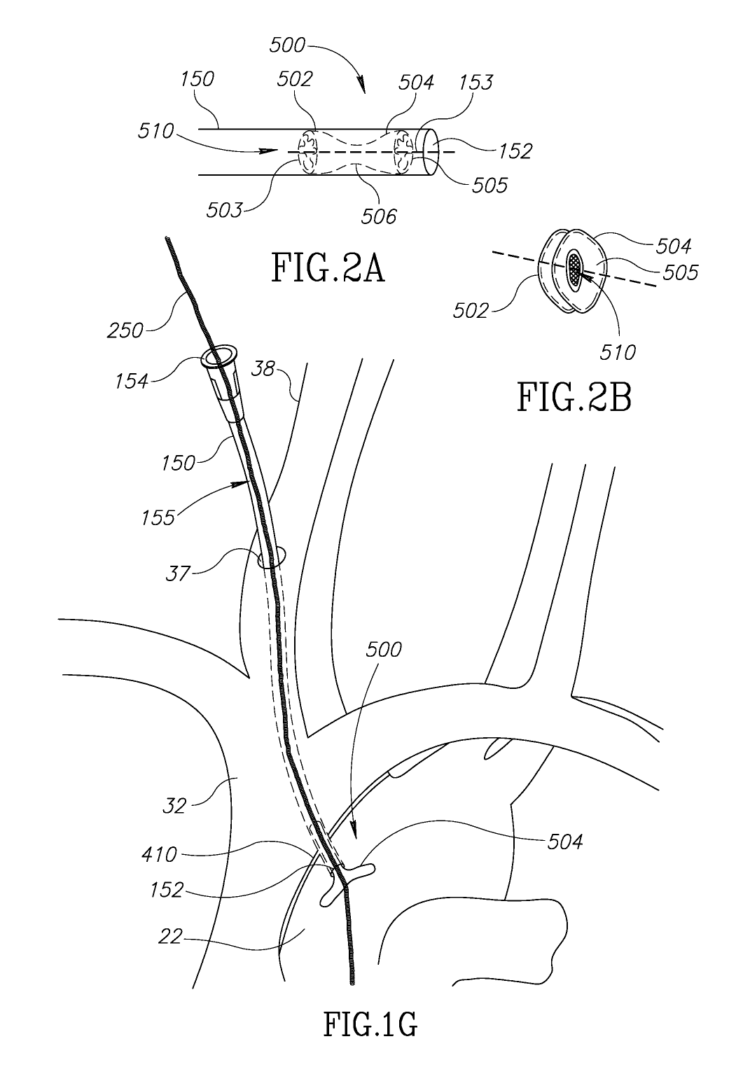

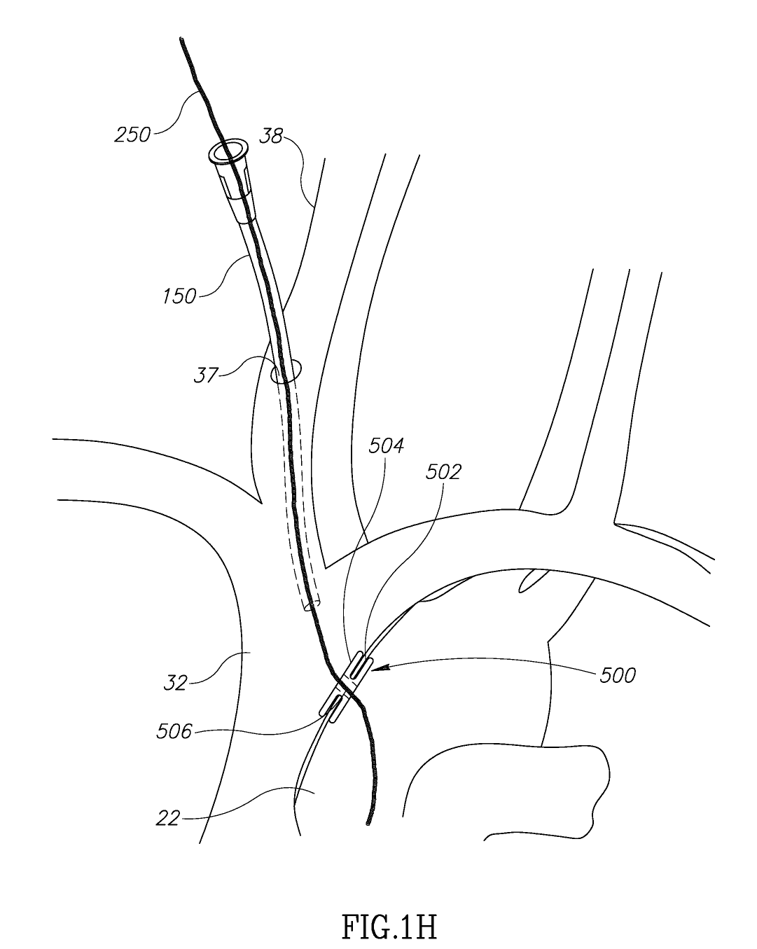

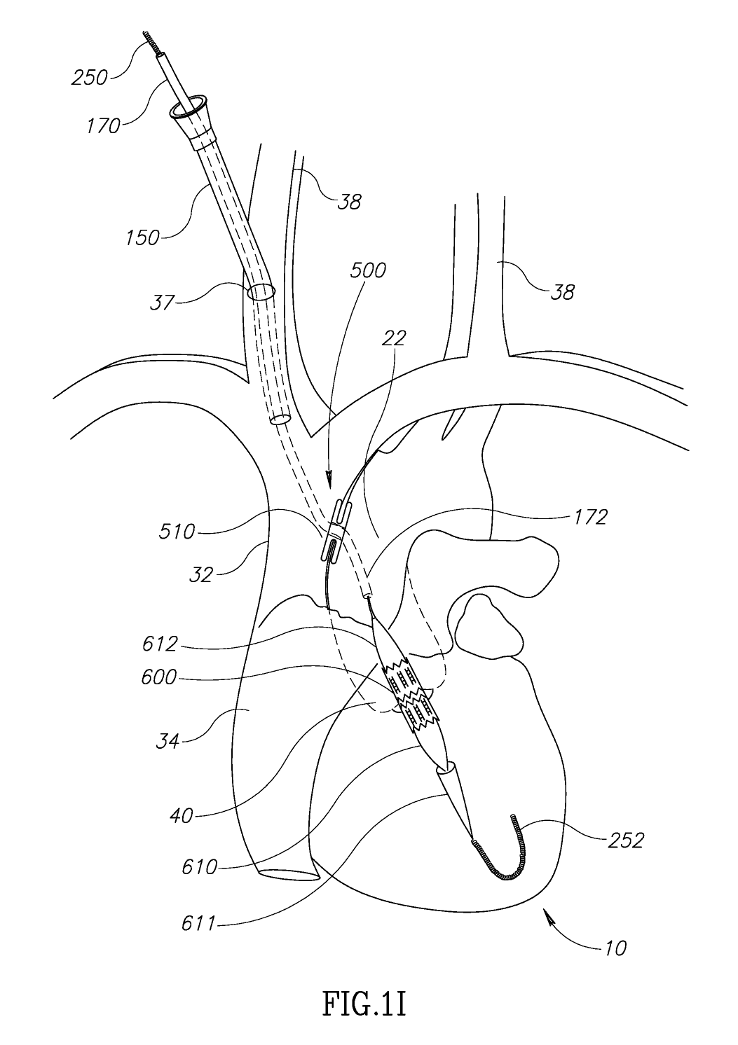

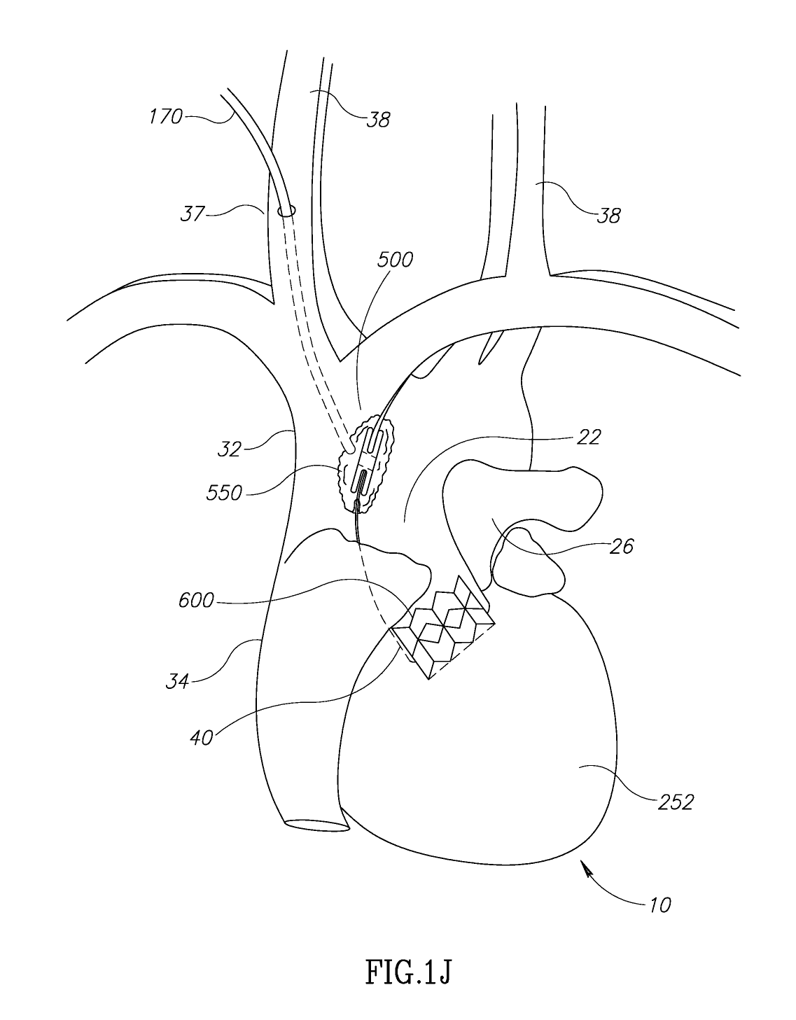

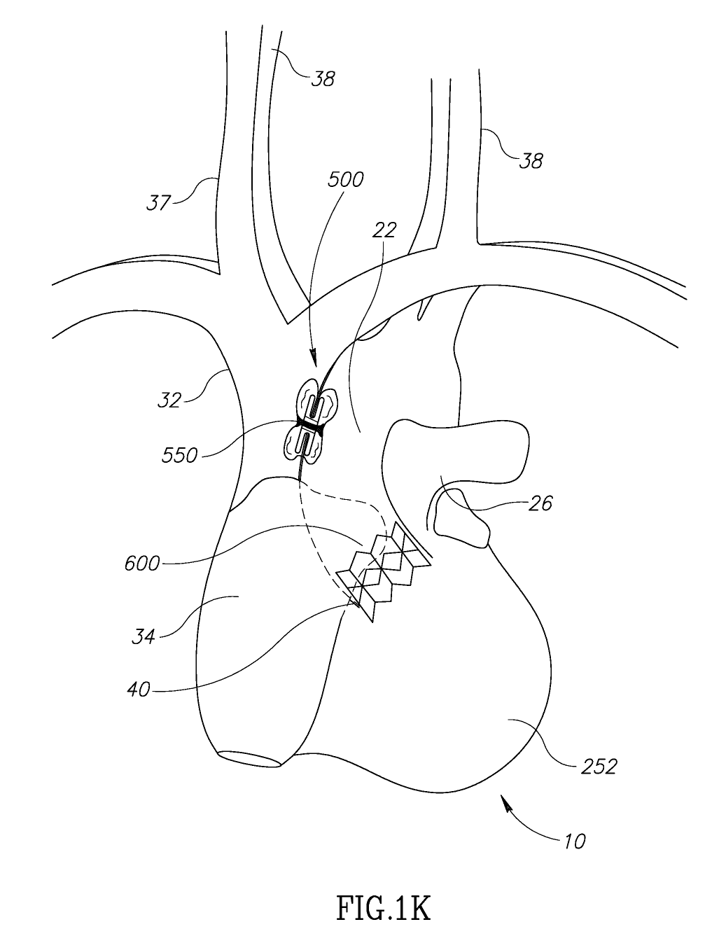

FIGS. 1A-1K schematically show a 3T-AVI method of deploying a PAV into a human heart to replace a native aortic valve with the PAV, in accordance with an embodiment of the disclosure. As schematically shown in FIGS. 1A-1F, a 3T-AVI method in accordance with an embodiment of the disclosure may comprise a first (“puncture”) phase in which a puncture is made in the SVC and the AA to create an access to the AA and the aortic valve from the SVC. As schematically shown in FIGS. 1G-1H, a 3T-AVI method in accordance with an embodiment of the disclosure may comprise a second (“approximation”) phase, in which the puncture between the SVC and the AA is secured and protected from further damage. As schematically shown in FIGS. 1I-1K, a 3T-AVI method in accordance with an embodiment of the disclosure may comprise a third (“deployment”) phase, in which the PAV is deployed in the native aortic valve.

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended. Unless otherwise indicated, the word “or” in the description and claims is considered to be the inclusive “or” rather than the exclusive or, and indicates at least one of, or any combination of items it conjoins.

FIGS. 1A-1B shows a schematic illustration of a human heart 10 showing AA 22, SVC 32, IVC 34, and pulmonary artery 26. Left ventricle (not shown) and AA 22 communicate via the aortic valve (not indicated in FIG. 1A-1B). Right ventricle (not shown) and pulmonary artery 26 communicate via the pulmonary valve (not shown). FIG. 1A also shows some peripheral veins, including subclavian vein 36 and jugular vein 38.

In the 3T-AVI method in accordance with an embodiment of the disclosure, a guidewire 200 threaded through a first sub-tube of a compound tube 100 is inserted via an opening 37 into jugular vein 38 and guided through SVC 32 to IVC 34 or into the right ventricle through the tricuspid valve. Guidewire 200 comprises a rounded end 202 to prevent inadvertent punctures. Compound tube 100 has a distal end 102 and proximal end 104 and guidewire 200 is used to guide compound tube 100 into SVC 32 through jugular vein 38. FIG. 1B schematically shows distal end 102 of compound tube 100 after being inserted into jugular vein 38 and guided to SVC 32 by guidewire 200.

As shown in FIG. 1B, a balloon 300, which is pre-loaded, deflated, into sub-tube 110 near distal end 102 of compound tube 100, is ejected from distal end 102 once the distal end is placed in lumen 33 of SVC 32.

Reference is now made to FIG. 1C, which schematically shows balloon 300 inflated within lumen 33 of SVC 32. In an embodiment of the disclosure, in addition to sub-tube 110, compound tube 100 comprises two additional sub-tubes 120 and 130, and distal end 102 of the compound tube comprises three ducts 112, 122, 132, each duct comprising a distal end of sub-tube 110, 120 and 130, respectively. In an embodiment of the disclosure, each of ducts 112, 122 and 132 are connected to a different portion of balloon 300, as described hereinbelow.

In an embodiment of the disclosure, balloon 300 is shaped so that once inflated, the balloon is shaped as a tube having a curved wall 302 comprising an outer surface 304 and an inner surface 306, the inner surface surrounding through hole 310 that traverses balloon 300 between a first end 312 of the balloon and a second end 314 of the balloon.

In an embodiment of the disclosure, balloon 300 further includes additional through holes, which may be referred to herein as “ports”, that traverse inside curved wall 302. In an embodiment of the disclosure, balloon 300 comprises a guidewire port 320 that traverses curved wall 302 between a first end 312 and a second end 314. In an embodiment of the disclosure, balloon 300 comprises a puncture port 322 that traverses curved wall 302 between first end 312 and outer surface 304. In an embodiment of the disclosure, balloon 300 further includes an inflation input (not show) for the balloon to receive a liquid (for example saline) or a gas to inflate the balloon.

In an embodiment of the disclosure, first sub-tube 110 is connected to guidewire port 320 at first end 312 of balloon 300 via duct 112, sub-tube 120 is connected to puncture port 322 at the first end of the balloon via duct 122, and sub-tube 130 is connected via duct 132 to the inflation input (not shown) at the first end of the balloon.

In an embodiment of the disclosure, balloon 300 in the inflated state is dimensioned so that the balloon's length, as measured between first end 312 and second end 314, is between 15 millimeters (mm) and 50 mm. In an embodiment of the disclosure, balloon 300 may be shaped so that the balloon as inflated has a substantially circular cross section along outer surface 304. Optionally, the circular cross section may be characterized by a diameter of between 15 and 35 mm. Optionally, the diameter of the cross section of balloon 300 may be different at different points along the length of balloon 300. By way of example, as shown in FIGS. 1C-1E, a middle portion of balloon 300 equidistant from first end 312 and second end 314 may have a cross section characterized by a larger diameter than a portion at or near the first or second ends. In an embodiment of the disclosure, through hole 310 may be characterized by a diameter that is between 15 and 30 mm. In an embodiment of the disclosure, the thickness of curved wall 302 may be between 0.05 mm and 2 mm.

Once balloon 300 is positioned within lumen 33 of SVC 32 at a region (schematically indicated with dashed oval 800) where SVC 32 is adjacent to AA 22, balloon 300 is inflated, by introducing a liquid into the balloon through sub-tube 130. In an embodiment of the disclosure, balloon 300 is sufficiently inflated to stabilize the location and orientation of the balloon in the lumen of SVC 32, and allow blood flow through the SVC via through hole 310. Before and or during inflation of balloon 300, the balloon is oriented so that an outer wall opening 323 of puncture port 322 faces AA 22, so that a needle ejected from puncture port 322 will be ejected towards AA 22 (see FIG. 1D).

Reference is now made to FIG. 1D. In an embodiment of the disclosure, after balloon 300 is positioned within lumen 33 of SVC 32 and inflated, and outer wall opening 323 of puncture port 322 is oriented to face AA 22, a needle 400 is ejected from outer wall opening 323 to create a puncture represented by a circle 410 that traverses wall 31 of SVC 32 and a wall 21 of AA 22 and connects lumen 33 of SVC 32 with lumen 23 of AA 22.

As shown in FIG. 1D, needle 400 is optionally loaded into compound tube 100 by inserting a delivery tube 450 into sub-tube 120, and inserting needle 400 connected to a control tube 402 into a proximal end 452 of a delivery tube 450, until a tip 404 of needle 400 reaches wall opening 323. Control tube 402 is then made to advance further so that the tip 404 of needle 400 extends outward from outer wall opening 323 of balloon 300 and pierces through wall 31 of SVC 32 and wall 21 of AA 22 into lumen 23 of the AA, thus creating puncture 410.

Reference is now made to FIGS. 1E and 1F. After puncture 410 is made, needle 400 and control tube 402 are withdrawn from sub-tube 120. Delivery tube 450 is kept within sub-tube 120, and a second guidewire 250 is inserted into delivery tube 450 and distal end 252 of guidewire 250 is made to advance out of outer wall opening 323 of balloon 300 and through puncture 410 into lumen 23 of AA 22, and through the aortic valve (not indicated in FIGS. 1E and 1F) into left ventricle 12 (FIG. 1F). After insertion of guidewire 250 into heart 10 is completed, with distal end 252 in left ventricle 12, delivery tube 450 is withdrawn from compound tube 100. Additionally, balloon 300 is deflated and withdrawn out of opening 37 together with guidewire 200 and compound tube 100, and optionally, as shown in FIG. 1F, only guidewire 250 remains inserted in jugular vein 38 and heart 10.

FIGS. 2A-2B schematically illustrates an approximation device (AD) 500 used for an approximation phase of a 3T-AVI method schematically illustrated in FIGS. 1G-1H following, as shown in FIG. 1F, removal of compound tube 100 in accordance with an embodiment of the disclosure.

FIG. 2A shows AD 500 in a collapsed state and loaded in a distal end 152 of a deployment tube 150, and FIG. 2B shows AD 500 in an expanded state once ejected from deployment tube 150.

As schematically shown in FIGS. 2A-2B, AD 500 in accordance with an embodiment of the disclosure AD 500 has a longitudinal axis 153 shown in a dashed line, and comprises a venous anchor 502 and an aortic anchor 504 connected by a bridge 506. In an embodiment of the disclosure AD 500 is shaped so that venous anchor 502 is formed having a first opening 503 and aortic anchor 504 is formed having a second opening 505. AD 500 in accordance with an embodiment of the disclosure is formed having a channel 510 traversing both anchors and tubular bridge 506 and connecting first opening 503 and second opening 505. In an embodiment of the disclosure, bridge 506 is shaped and dimensioned to be narrow relative to venous anchor 502 and aortic anchor 504 when AD is in the expanded state.

In an embodiment of the disclosure, at least one of venous anchor 502, aortic anchor 504 and bridge 506 comprises a wire mesh formed from a shape memory material, optionally comprising nitinol. In an embodiment of the disclosure, channel 510 of AD 500 in the collapsed state has a diameter between 3 mm and 12 mm, which is equivalent to between 9 Fr (French gauge units) and 36 Fr. In an embodiment of the disclosure, channel 510 of AD 500 in the expanded state has a diameter sufficient to enable passage of a deployment tube used to deploy an artificial aortic valve, which may be less than about 10 mm.

Puncture 410 produced in a puncture phase of a 3T-AVI method in accordance with an embodiment of the disclosure advantageously creates an unobstructed access path from jugular vein 38, to AA 22. In an embodiment of the disclosure, the 3TA-AVI method may comprise an approximation phase in which puncture 410 is augmented and protected from further damage by AD 500. FIGS. 1G-1H schematically illustrate the approximation phase in accordance with an embodiment of the disclosure.

In an embodiment of the disclosure, after guidewire 250 is successfully inserted in heart 10, deployment tube 150 is inserted into the subject. Optionally, proximal end 154 of deployment tube 150 comprises a valve allowing insertion of wires and catheters into the deployment tube as needed while preventing flow of blood out of the deployment tube. In an embodiment of the disclosure, deployment tube 150 is pre-loaded with AD 500 in a collapsed state. Deployment tube 150 is inserted into the subject with guidance provided by guidewire 250, which passes through a lumen 155 of deployment tube 150 and channel 510 (FIGS. 2A, 2B) of collapsed AD 500.

Distal end 152 of deployment tube 150 is advanced through opening 37 of jugular vein 38, SVC 32, and puncture 410 into AA 22. As shown in FIG. 1G, once distal end 152 is positioned in AA 22, aortic anchor 504 is ejected from distal end 152 and the aortic anchor expands from the collapsed state to the expanded state.

Once aortic anchor 504 is ejected and expanded to its expanded state, deployment tube 150 is withdrawn further while guidewire 250 is kept in place. Expanded aortic anchor 504 is shaped and dimensioned to be unable to pass through puncture 410 and is kept inside AA 22. And as deployment tube 150 is withdrawn further through puncture 410 back into SVC 32 bridge 506 is ejected from deployment tube 150 to seat inside puncture 410 (FIG. 1G). As deployment tube 150 is withdrawn further back towards jugular vein 38, venous anchor 502 is ejected from deployment tube 150 to expand on the SVC 32 side of puncture 410. The anchoring provided by aortic anchor 504 and venous anchor 502 prevents inadvertent dislodging of AD 500 from puncture 410, and channel 510 maintains, protects, and optionally widens puncture 410 in order to expedite deployment of a desired apparatus into AA 22 and/or aortic valve 40 from a peripheral vein.

Reference is now made to FIGS. 1I-1J. After AD 500 is deployed within puncture 410, a distal end 172 of a PAV tube 170 preloaded with a PAV 600 in a collapsed state is advanced, guided by guidewire 250, through deployment tube 150 and channel 510 into AA 22. PAV 600 in a partially collapsed state is ejected from PAV tube 170. PAV 600 may be capped and prevented from fully expanding by a front nosecone 610 and a back nosecone 612. PAV 600 may be loaded with a second front nosecone 611 to further facilitate traversal through channel 510 and aortic valve 40. Once partially expanded, PAV 600 is positioned within aortic valve 40, nosecones 610 and 612 are dislodged from PAV 600 so that the PAV expands into a fully expanded state as schematically shown in FIG. 1J, and is deployed in aortic valve 40. Once PAV 600 is deployed in aortic valve 40, nosecones 610, 612 (FIG. 1I) are withdrawn via guidewire 250, as schematically shown in FIG. 1K.

Following deployment of PAV 600, channel 510 of AD 500 is plugged with a plug 550, which by way of example may be an Amplatzer Occluder Device. In an embodiment of the disclosure, plug 550 is loaded into deployment tube 150 and inserted into channel 510 via distal end 152 of the deployment tube, guided by guidewire 250.

It is noted that whereas in the above discussion access to the AA from the SVC is described as being used by way of example to replace an aortic valve, practice of embodiments of the disclosure are not limited to replacement of aortic valves, and embodiments of the disclosure may be used to facilitate many different procedures. For example, access to the aorta in accordance with an embodiment of the disclosure enables access to the left ventricle and via the left ventricle to the mitral valve and left atrium for performance of procedures at any of these sites.

Nor is practice of embodiments of the disclosure limited to procedures involving access to the aorta via the SVC and the disclosure provides a method of accessing a first lumen delimited by a first wall from a second adjacent lumen delimited by a second wall, the method comprising: guiding an inflatable balloon housed in a first tube via a first guidewire to a region of the second lumen for which a region of the second wall is adjacent to a region of the first wall; inflating the balloon so that a needle port comprised on an outer surface of the balloon is securely positioned on an interior side of the region of the second wall facing the region of the first wall; and extending a needle through the needle port to create a puncture traversing the regions of the second and first walls to provide access to the first lumen from the second lumen.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Descriptions of embodiments of the disclosure in the present application are provided by way of example and are not intended to limit the scope of the disclosure. The described embodiments comprise different features, not all of which are required in all embodiments. Some embodiments utilize only some of the features or possible combinations of the features. Variations of embodiments of the disclosure that are described, and embodiments comprising different combinations of features noted in the described embodiments, will occur to persons of the art. The scope of the invention is limited only by the claims.

1. The method according to claim 21 comprising:
guiding an inflatable balloon housed in a first tube via a first guidewire to a portion of the superior vena cava (SVC) that is adjacent to a region of the wall of the ascending aorta (AA);
inflating the balloon so that a needle port comprised on an outer surface of the balloon is securely positioned on an interior side of a wall of the SVC portion facing the region of the wall of the wall of the AA; and
extending a needle through the needle port to create a puncture traversing the SVC portion wall and the region of the wall of the AA that enables access to the AA from the SVC.

2. The method according to claim 1 and comprising inserting a second guidewire from the SVC through the balloon and the puncture into the AA.

3. The method according to claim 1, the method further comprising removing the first tube, balloon and first guidewire from the SVC.

4. The method according to claim 2 the method further comprising:
guiding via the second guidewire a distal end of a second tube from the SVC through the puncture into the AA, the second tube having an approximation device (AD) loaded in a collapsed state near the distal end; and
withdrawing the second tube to eject the AD so that it expands and clamp the walls of the SVC and AA between an aortic anchor and a venous anchor connected by a bridge that forms a channel having first and second openings in the SVC and AA respectively.

5. The method according to claim 4 further comprising:
guiding via the second guidewire a third tube having a collapsed prosthetic aortic valve (PAV) loaded near its distal end from the SVC through the puncture and into the AA to position the distal end in a vicinity of an antegrade side of the aortic valve; and
ejecting the PAV from the distal end at the native aortic valve, to expand and replace the native aortic valve.

6. The method according to claim 5, further comprising:
withdrawing the third tube from the AA and the puncture; and
closing the channel formed by the bridge.

7. The method according to claim 6 wherein closing the channel comprises seating a plug in the channel.

8. The method according to claim 1, wherein in an inflated state the balloon seals to the SVC portion to prevent blood flow between the exterior of the balloon and the interior wall of the SVC.

9. The method according to claim 8, wherein the balloon in the inflated state the balloon is formed having a through hole that enables blood flow through the SVC.

10. The method according to claim 8 wherein in the inflated state the balloon is barrel shaped having a larger cross section in a region of a middle of the balloon than cross sections near its respective ends.

11. The method according to claim 1 comprising inserting an approximation device (AD) into the puncture to maintain contact between the wall of the SVC portion and the wall of the region of the AA, the AD having a through-hole via which the AA is accessible from the SVC.

12. An approximation device (AD) comprising:
a cylindrical wall comprising a waist region between two end regions the wall defining a lumen having a longitudinal axis and first and second open ends with which the lumen communicates, the wall having a collapsed and expanded state;
wherein in an expanded state the cylindrical wall assumes a shape having a relatively narrow waist region that expands out to a relatively large region at each end, the narrow waist characterized by a radial dimension perpendicular to the longitudinal axis and each large region characterized by a respective radial dimension substantially larger than the radial dimension characterizing the waist, and in the collapsed state assumes a shape for which the end regions have respective radial dimensions substantially smaller than the respective radial dimensions they assume in the expanded state.

13. The approximation device according to claim 12 wherein in the collapsed state the radial dimensions of the waist and end regions is less than or equal to about 12 mm (millimeters).

14. The approximation device according to claim 13 wherein in the collapsed state the radial dimensions of the waist and end regions is less than or equal to about 6 mm.

15. The approximation device according to claim 13 wherein in the expanded state the radial dimension of the waist region is about equal to the radial dimension of the waist region in the collapsed state.

16. The approximation device according to claim 13 wherein in the expanded state the radial dimension of an end region is greater than about 15 mm.

17. The approximation device according to claim 16 wherein in the expanded state the radial dimension of an end region is greater than about 20 mm.

18. (canceled)

19. An inflatable balloon configured for insertion into a blood vessel, the balloon comprising:
a tubular wall comprising an outer surface and an inner surface, the inner surface surrounding a first through hole having a first opening at a first end of the balloon and a second opening at a second end of the balloon; and
a second through hole traversing a portion of the tubular wall, the second through hole formed having a first opening at the first end and a second opening at the outer surface.

20. The inflatable balloon according to claim 19 further shaped having a third through hole traversing a portion of the tubular wall, the third through hole formed having a first opening at the first end and a second opening at the second end.

21. A method of providing access to the ascending aorta (AA) via the superior vena cava (SVC) comprising puncturing adjacent wall regions of the SVC and AA to create aligned holes in the adjacent wall regions that provide passage from the SVC into the AA.
